(19) United States
(12) Patent Application Publication
(10) Pub. No.: US 2025/0079003 A1
(43) Pub. Date: Mar. 6, 2025

(54) AUTOMATED SYSTEMS FOR DIAGNOSIS AND MONITORING OF STROKE AND RELATED METHODS

(71) Applicant: Ohio State Innovation Foundation, Columbus, OH (US)

(72) Inventors: Alper YILMAZ, Lewis Center, OH (US); Deepak Kumar GULATI, Columbus, OH (US)

(21) Appl. No.: 18/726,529

(22) PCT Filed: Jan. 5, 2023
(86) PCT No.: PCT/US2023/060148

§ 371 Date: Jul. 3, 2024

Related U.S. Application Data
(60) Provisional application No. 63/296,602, filed on Jan. 5, 2022.

(51) Int. Cl. G16H 50/20; A61B 5/00; G06V 40/16; G10L 25/66; G16H 40/67; G16H 50/30

(52) U.S. Cl. CPC G16H 50/20; A61B 5/0077; A61B 5/4803; A61B 5/7267; A61B 5/7282; G06V 40/174; G10L 25/66; G16H 40/67; G16H 50/30; A61B 2562/0204; A61B 2576/00

(57) ABSTRACT

An example system for detecting stroke includes an imaging device; a microphone; and a computing device. The computing device is configured to receive a sequence of images capturing a state of a patient; analyze the sequence of images to detect one or more of limb impairment, gaze impairment, or facial palsy; and assign a respective numeric score to each of the detected one or more limb impairment, gaze impairment, or facial palsy. The computing device is also configured to receive an audio signal capturing a voice of the patient; analyze the audio signal to detect aphasia or agnosia; assign a respective numeric score to the detected aphasia or agnosia. The computing device is further configured to generate a stroke score, which is a sum of the respective numeric scores for the detected one or more of limb impairment, gaze impairment, or facial palsy and the detected aphasia or agnosia.

### CROSS-REFERENCE TO RELATED APPLICATIONS

This application claims the benefit of U.S. provisional patent application No. 63/296,602, filed on Jan. 5, 2022, and titled “AUTOMATED SYSTEMS FOR DETECTION OF STROKE AND RELATED METHODS,” the disclosure of which is expressly incorporated herein by reference in its entirety.

### BACKGROUND

Stroke is a medical emergency. The annual incidence of new or recurrent strokes in the United States (US) is nearly 800,000. More than 140,000 people die annually in the US from strokes, which makes it the fifth leading cause of death in US and second most common cause of death across the globe. Stroke also remains a significant cause of serious long-term disability. In the US alone, the estimated annual costs of stroke are $40 billion and are projected to triple by 2030. Endovascular thrombectomy (EVT, mechanical removal of clot) has recently revolutionized the stroke care in 2019 by providing new modality of treatment as well as by extending the treatment window to 24 hours. The speed of treatment delivery remains critical. Every delay in delivering EVT has a fundamental negative impact on the patient outcomes.

To access stroke severity and early detection, there are prehospital stroke scales, such as Rapid Arterial oCclusion Evaluation (RACE) primarily used in a pre-hospital setting by paramedics. RACE scale is a 5-item validated scale from 0 to 11 and considers facial palsy, arm motor impairment, leg motor impairment, gaze deviation and hemiparesis to identify stroke patients with large vessel occlusion (LVO). Implementation of RACE and other stroke scales constantly require training of emergency medical responders and needs extensive financial and personnel resources. Additional challenges with the current manual evaluation of stroke scales include inter-observer variability in assessment and different types of biases in the field that could potentially delay stroke treatment.

### SUMMARY

Described herein is a technology to automate and standardize calculation of stroke scales, such as RACE, for early detection of stroke with LVO through audio-video interface with the patient. The technology includes a user friendly artificial intelligence (AI) agent which can be used in the field (e.g., prehospital—ambulances, rural hospitals), triage area of the ER or in-home care settings to create stroke alerts that eliminates the delay in providing time sensitive treatment to patients with stroke.

The technology can detect and track facial features and body parts to evaluate facial palsy and motor impairment of arms and leg, evaluate verbal response for any speech or language impairment, as well as analyze touch sensing. The system uses machine learning to understand the motion of the body and facial parts with the touch sensing capabilities for healthy and stroke patients to create the score for each item in the scale independent of the patient gender and age.

An example system for detecting stroke includes an imaging device; a microphone; and a computing device. The computing device is configured to receive a sequence of images from the imaging device, the sequence of images capturing a state of a patient; analyze the sequence of images to detect one or more of limb impairment, gaze impairment, or facial palsy; and assign a respective numeric score to each of the detected one or more limb impairment, gaze impairment, or facial palsy. The computing device is also configured to receive an audio signal from the microphone, the audio signal capturing a voice of the patient; analyze the audio signal to detect aphasia or agnosia; assign a respective numeric score to the detected aphasia or agnosia. The computing device is further configured to generate a stroke score, which is a sum of the respective numeric scores for the detected one or more of limb impairment, gaze impairment, or facial palsy and the detected aphasia or agnosia.

In some implementations, the computing device is further configured to diagnose the patient with a stroke based on the stroke score. Optionally, the computing device is further configured to assess a severity of the stroke based on the stroke score. Alternatively or additionally, the computing device is optionally further configured to recommend a triage action. For example, the recommended triage action is selecting a treatment facility. Alternatively or additionally, the computing device is optionally further configured to recommend a treatment for the patient. For example, the recommended treatment is administration of a thrombolytic or performance of an endovascular procedure.

In some implementations, the step of analyzing the sequence of images includes using a machine learning model.

Alternatively or additionally, in some implementations, the step of analyzing the audio signal includes using a machine learning model.

Alternatively or additionally, in some implementations, the step of assigning a respective numeric score includes using a machine learning model. Alternatively, in some implementations, the step of assigning a respective numeric score includes using an expert system.

In some implementations, the system further includes an expert system. Optionally, the expert system is configured to analyze the sequence of images, analyze the audio signal, assign the respective scores, and/or generate the stroke score.

In some implementations, the system further includes a trained machine learning model, where the trained machine learning model is configured to analyze the sequence of images, analyze the audio signal, assign the respective scores, and/or generate the stroke score.

In some implementations, the system further includes a haptic device configured to apply a force, a vibration, or a motion to the patient. The computing device is further configured to control the haptic device.

Alternatively or additionally, the stroke score is a Rapid Arterial oCclusion Evaluation (RACE), National Institutes of Health Stroke Score (NIHSS), Los Angeles Motor Scale (LAMS), or Cincinnati Stroke Scale.

Alternatively or additionally, the system is a smart phone, a tablet computer, a laptop computer, or a desktop computer.

Another system for detecting stroke includes one or more artificial intelligence (AI) models; and a computing device. The computing device is configured to receive a sequence of images, the sequence of images capturing a state of a patient; receive an audio signal, the audio signal capturing a voice of the patient; input the sequence of images and the audio signal into the one or more AI models; and receive a stroke score, the stroke score being predicted by the one or more AI models.

Optionally, the computing device is further configured to extract one or more features from the sequence of images and the audio signal, and the step of inputting the sequence of images and the audio signal into the one or more AI models includes inputting the extracted features into the one or more AI models. Optionally, the one or more AI models are an expert system. Alternatively or additionally, the one or more AI models include one or more trained machine learning models.

In some implementations, the one or more trained machine learning models include a transformer neural network (TiN). Alternatively or additionally, the one or more trained machine learning models include a multilayer perceptron (MLP).

In some implementations, the one or more trained machine learning models are trained using a reinforcement learning strategy.

In some implementations, the system optionally further includes an imaging device for capturing the sequence of images and a microphone for capturing the audio signal.

In some implementations, the computing device is further configured to diagnose the patient with a stroke based on the stroke score. Optionally, the computing device is further configured to assess a severity of the stroke based on the stroke score. Alternatively or additionally, the computing device is optionally further configured to recommend a triage action. For example, the recommended triage action is selecting a treatment facility. Alternatively or additionally, the computing device is optionally further configured to recommend a treatment for the patient. For example, the recommended treatment is administration of a thrombolytic or performance of an endovascular procedure.

An example computer-implemented method for automated detection of stroke is also described herein. The example computer-implemented method includes: receiving, from an imaging device, a sequence of images, the sequence of images capturing a state of a patient; analyzing the sequence of images to detect one or more of limb impairment, gaze impairment, or facial palsy; assigning a respective numeric score to each of the detected one or more limb impairment, gaze impairment, or facial palsy; receiving, from a microphone, an audio signal, the audio signal capturing a voice of the patient; analyzing the audio signal to detect aphasia or agnosia; assigning a respective numeric score to the detected aphasia and/or agnosia; and generating a stroke score including a sum of the respective numeric scores for the detected one or more of limb impairment, gaze impairment, or facial palsy and the detected aphasia or agnosia.

In some implementations, the computer-implemented method further includes diagnosing the patient with a stroke based on the stroke score. Optionally, the computer-implemented method further includes assessing a severity of the stroke based on the stroke score.

In some implementations, the computer-implemented method further includes recommending a triage action.

In some implementations, the computer-implemented method further includes recommending a treatment for the patient.

In some implementations, the step of analyzing the sequence of images includes using a machine learning model.

In some implementations, the step of analyzing the audio signal includes using a machine learning model.

In some implementations, the step of assigning a respective numeric score includes using a machine learning model.

In some implementations, the step of assigning a respective numeric score includes using an expert system.

In some implementations, the stroke score is a Rapid Arterial oCclusion Evaluation (RACE), National Institutes of Health Stroke Score (NIHSS), Los Angeles Motor Scale (LAMS), or Cincinnati Stroke Scale.

An example computer-implemented method for automated monitoring of a medical condition is described herein. The example computer-implemented method includes: receiving, from an imaging device, a sequence of images, the sequence of images capturing a state of a patient; receiving, from a microphone, an audio signal, the audio signal capturing a sound associated with the patient; extracting a plurality of features from the sequence of images and the audio signal; inputting the extracted features into one or more artificial intelligence (AI) models; and monitoring, using the one or more AI models, a medical condition of the patient.

In some implementations, the one or more AI models are an expert system.

In some implementations, the one or more AI models include one or more trained machine learning models.

In some implementations, the medical condition is seizure, pain management, or paralysis.

In some implementations, the computer-implemented method further includes at least one of diagnosing the medical condition based on an output of the one or more AI models, recommending a triage action based on the output of the one or more AI models, or recommending a treatment based on the output of the one or more AI models.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates detecting and tracking facial features and body parts to evaluate facial palsy and motor impairment of arms and leg according to implementations described herein.

FIGS. 2A-2D are diagrams illustrating functionality of one or more sub-systems for evaluating a patient for stroke according to implementations described herein. FIG. 2A illustrates arm motor impairment scoring. FIG. 2B illustrates facial palsy scoring. FIG. 2C illustrates gaze deviation scoring. FIG. 2D illustrates head deviation scoring.

FIG. 3A is a diagram illustrating an example machine learning based pipeline for evaluating a patient for stroke according to implementations described herein. FIG. 3B is a diagram illustrating another example machine learning based pipeline for evaluating a patient for stroke according to implementations described herein.

FIG. 4 is an example computing device.

FIG. 5 is a diagram that illustrates an example system for outputting a stroke scale using an AI model, according to implementations described herein.

FIG. 6 illustrates example operations for automated detection of a stroke, according to implementations described herein.

FIG. 7 illustrates example operations for automated monitoring of a medical condition, according to implementations described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for automated analysis (e.g., using AI) of images and/or audio associated with a patient to generate a stroke score, it will become evident to those skilled in the art that the implementations are not limited thereto, but are applicable for automated analysis of images and/or audio associated with a patient to monitor for other medical conditions (e.g., seizures, motor disorders, pain, etc.).

As used herein, the terms “about” or “approximately” when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable means for delivering the agent. Administration includes self-administration and the administration by another.

The term “subject” (sometimes referred to a “patient” herein) is defined herein to include animals such as mammals, including, but not limited to, primates (e.g., humans), cows, sheep, goats, horses, dogs, cats, rabbits, rats, mice and the like. In some implementations, the subject is a human.

This disclosure contemplates that the systems and methods described herein can be used in different settings. For example, the systems and methods described herein may be employed in an emergency room (ER) and/or urgent care setting. In these settings, the systems and methods described herein are used to diagnose and/or assess the severity of a stroke. It should be understood that more comprehensive stroke scales (e.g., NIHSS) may be used in these settings. The systems and methods described herein can also be employed in hospital (e.g., hospital systems), pre-hospital (e.g., EMS, schools, athletic events, etc.), nursing home, post-hospital (e.g., health insurance, public use, etc.), or in-home settings, for example, to monitor a patient. Optionally, the systems and methods are used in these settings for remote monitoring of the patient. It should be understood that less comprehensive stroke scales may optionally be used in these settings.

The effectiveness of stroke care can depend on rapidly detecting the stroke (and optionally assessing its severity) and transporting a stroke patient to a facility with the best available stroke treatment technology and personnel. For example, there is a critical window (e.g., 3 hours) in which to diagnose stroke in an ER setting and take appropriate actions. Strokes going undetected have negative consequences for quality of life. Detecting strokes rapidly, and/or distinguishing between types of strokes can therefore have a significant effect on improving outcomes. Healthcare providers can struggle to detect strokes quickly because different stroke measurements are used in different contexts. Current methods of detecting strokes depend on human training and are therefore vulnerable to errors caused by human error or training deficiencies. A common stroke detection scale is the NIH stroke scale, but the NIH stroke scale is designed for use in hospitals and relatively complicated to administer. Alternative stroke scales can be easier to administer, but can sacrifice reliability and do not correlate perfectly with the NIH stroke scale. As a result, healthcare providers in a community may be using several inconsistent stroke scales (e.g., at a hospital, nursing home, urgent care center, or primary care physician's office). Therefore, the systems and methods described herein provide technological solutions that can be used across different settings and in different locations. Additionally, the systems and methods described herein improve the uniformity, speed, and quality of stroke detection, allowing for uniform and high-quality stroke evaluations. Moreover, the systems and methods described herein provide solutions for post-hospital monitoring, which is not possible using conventional systems.

For example, the existing solution for stroke evaluation is manual and there are no automated tools for stroke detection in the market. There are wearable sensors to automatically detect stroke (e.g., https://news.samsung.com/global/c-lab-engineers-developing-wearable-health-sensor-for-stroke-detection) by analyzing brain waves. These solutions require the patient to wear a device on the head and wait up to 15 minutes collect brain waves for analysis. Another group of approaches use MEMS motion sensors to detect stroke. Similar to brainwave approach the motion sensor based approach requires the patient to wear the sensors. Another body of work considers interaction of the patient with pressure sensitive surfaces, such as touch screens and keyboards (e.g., Bat-Orgil Bat-Erdene, Jeffrey L. Saver. Automatic Acute Stroke Symptom Detection and Emergency Medical Systems Alerting by Mobile Health Technologies: A Review. Journal of Stroke and Cerebrovascular Diseases. Volume 30, Issue 7, July 2021). These solutions require the patient to interact with the device in person and cannot be used for many of the significant stroke symptoms which makes it difficult to use in hyper-acute time window. Aside from automation of stroke detection, literature has studies for assessment of individual elements used to detect stroke, such as facial palsy (e.g., Hyun Seok Kim, So Young Kim, Young Ho Kim, and Kwang Suk Park. A Smartphone-Based Automatic Diagnosis System for Facial Nerve Palsy. Sensors 2015, 15(10)), for non-stoke domains. Another body of work requires Computed Tomography imaging that can be only obtained after the patient is transferred to a medical facility with necessary equipment and experts that can interpret these images. There is therefore no existing solution that can detect stroke remotely or onsite without wearing devices or using expensive medical imaging modalities. The technology solution with AI agent described herein fills an unmet need. A table comparing the AI agent solution described herein to existing solutions is provided below.

| | AI | Brain | MEMS | CT | Human |
|---|---|---|---|---|---|
| Feature Matrix | Agent | Waves | sensors | Imaging | expert |
| Prehospital | ✓ | x | x | x | ✓ |
| Post hospital | ✓ | x | x | x | x |
| Untrained (e.g., non | ✓ | x | x | x | x |
| expert) personnel | | | | | |
| Can analyze language | ✓ | x | x | x | ✓ |
| Can analyze neglect | ✓ | x | x | x | ✓ |
| Can analyze sensory | ✓ | x | x | x | ✓ |
| input | | | | | |
| Standard across | ✓ | ✓ | ✓ | ✓ | x |
| applications | | | | | |

One common example of problems in the field of stroke detection and treatment is the errors in execution of the stroke protocols by emergency medical personnel (EMS) personnel causing millions of dollars lost to lawsuits against providers. These lawsuits are based on the incorrect stroke scaling performed by the paramedics resulting in going to the closer hospital instead of going to a facility that can perform EVT. The technology solution described herein will eliminate such cases. Another immediate impact of the technology solution described herein will be reduction in morbidity rates for stroke patients which increases the patient outcomes required for most hospitals.

The technology solution with AI agent described herein has one or more of the following features: (1) Arm and leg motor impairment analysis sub-system that builds on the stroke detection standards, (2) Facial analysis sub-system based on detection of the asymmetries in the face features as well as the pupils for cephalic deviation defined in stroke detection standards, (3) Speech and motor synchronization sub-system for herniparesis detection defined in stroke detection standards, and (4) Patient interface design integrating the three elements (motor, facial, speech) in stroke detection. In some implementations, the technology solution is provided as an application (e.g., mobile application), which can integrate with existing medical provider systems such as on-site cameras and microphones. In some implementations, the technology solution is provided as an add-on to existing medical provider systems. In some implementations, the technology solution is provided as a standalone system, e.g., self-service and self-contained for use in medical or non-medical settings.

Arm and leg motor impairment sub-system: The motor impairment sub-system uses computer vision (CV) technology to acquire sequence of images from the camera of the mobile platform and analyze the motion of hands and arm to estimate a stroke score. The score is added to the queue of scores that are generated from the other sub-systems. The results of the motor impairment sub-system can be verified by the stroke expert.

Facial analysis sub-system: The facial analysis sub-system uses computer vision (CV) technology to acquire a sequence of images and performs two tasks by detecting the pupils and other facial features of the face. The pupils and features are further analyzed to estimate two scores by quantifying asymmetries in the form of cephalic deviation of gaze and drooping or asymmetric muscle contractions. The two scores are added to the queue of scores that are generated from the other sub-systems. The results of the facial analysis sub-system can be verified by the stroke expert.

Aphasia/agnosia sub-system (also referred to as “speech and motor synchronization sub-system”): The aphasia/agnosia sub-system tests a patient for aphasia and agnosia respectively for right and left part of brain functionality. The sub-system analyzes the speech and motor synchronization. Particularly for aphasia detection the sub-system uses computer vision (CV) technology to monitor response to verbal orders given to the patient and analyzes the motor response to generate a score that is added to the queue of stroke scores. For agnosia analysis, the sub-system prompts delivery of multiple questions to the patient to detect asomatognosia or anosognosia through analysis of the patient's verbal response. The patient's response is analyzed to generate a stroke score that is also added to the queue of stroke scores. The results of the developed aphasia/agnosia sub-system can be verified by the stroke expert.

Interface design and sub-systems integration: The interface integrates the sub-systems described above and decides and displays the stroke scale. It can have a web based dashboard that updates the patient's stroke status and alerts the caregivers via a text message to expedite the caregiving in the case of a stroke. The interface can be validated by stroke experts, nurses, and EMS personnel.

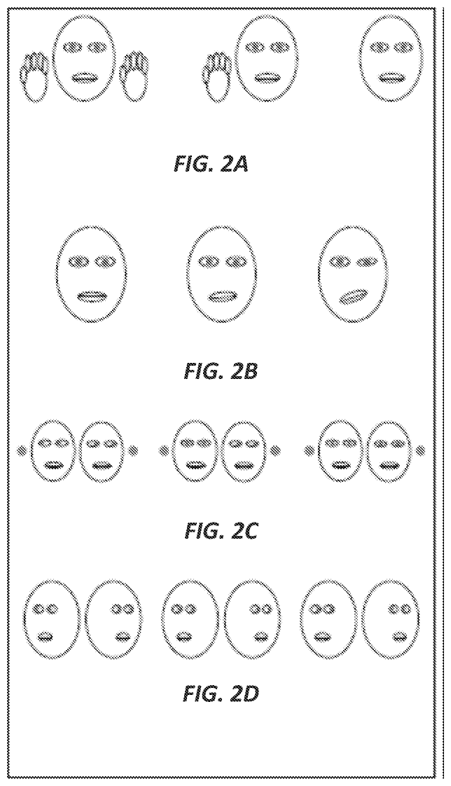

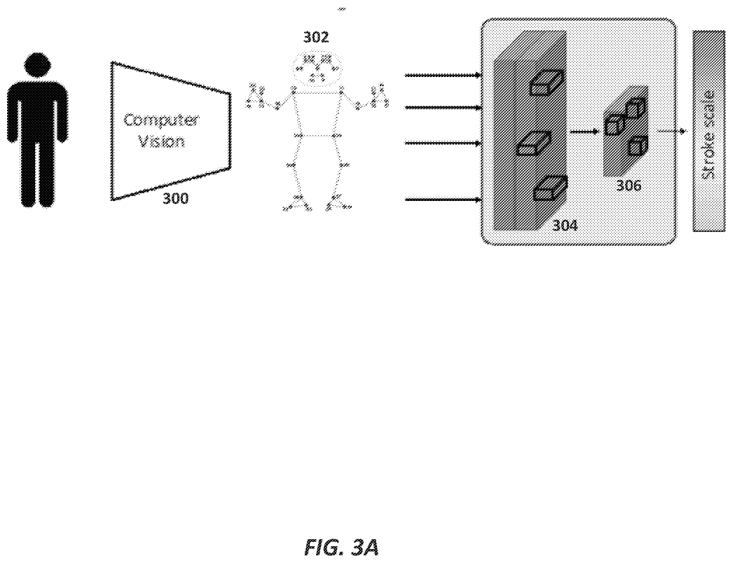

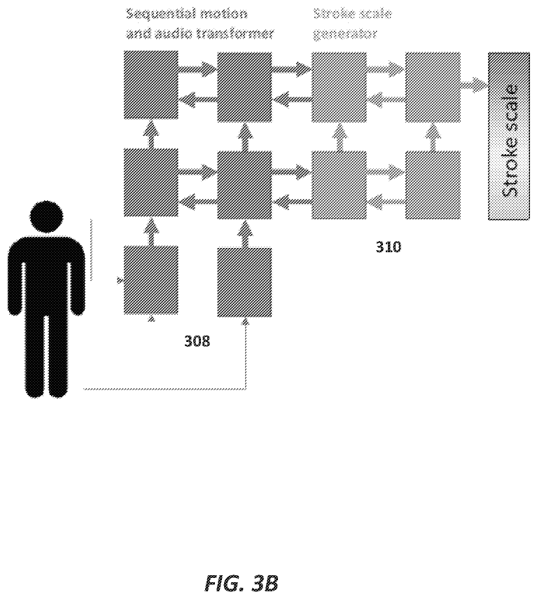

A prototype that computes stroke scores for arm motor impairment, facial palsy and gaze deviation and aphasia/agnosia has been developed. This is referred to herein as the pipeline based design. An example pipeline based design is illustrated by FIG. 3A. An illustration of rules the system can use to determine scores for presence or absence of a stroke as well as the severity during a stroke are shown in FIGS. 2A-2D. FIG. 2A illustrates arm motor impairment scoring based on the subject's ability to uphold both limbs—(left image) absent, (middle image) moderate stroke, (right image) severe stroke. FIG. 2B illustrates facial palsy scoring based on asymmetric face motion—(left image) absent, (middle image) moderate stroke, (right image) severe stroke. FIG. 2C illustrates gaze deviation scoring based on eye tracking of a target (e.g., dots shown in figure) to both sides—(left image) absent, (middle image) mild stroke, (right image) severe stroke. FIG. 2D illustrates head deviation scoring based on the subject's eyes following head rotation—(left image) absent, (middle image) mild stroke, (right image) severe stroke. The example implementation described herein can use a pipeline design which first detects and tracks body parts and facial features. Using the tracked facial features, the second part of the pipeline derives differential measures from the tracked positions in the images relative to other features to measure the stoke scale for each milestone. This information is modeled by AI using either an expert system (ES), decision trees (DT) or a neural network (NN). The ES and DT takes the motion and sensing information and generates scores through regression to alert the caregiver. The NN solution on the other hand performs linear combinations of the input variables in multitudes of neural layers to regress the stroke score. The differential measures consider the position, the speed and acceleration of the facial and body features as they move relative to others. In addition to the visual analysis, the pipeline also conducts speech analysis by prompting delivery of multiple questions to the patient and analyzing the correctness of the patient's response. The output of the second part of pipeline is the score for each milestone which are accumulated to provide the severity of the stroke on a 0-11 scale. This pipeline is generic and can accommodate other stroke scales such as the National Institutes of Health (NIH) Stroke scale which ranges from 0 to 42.

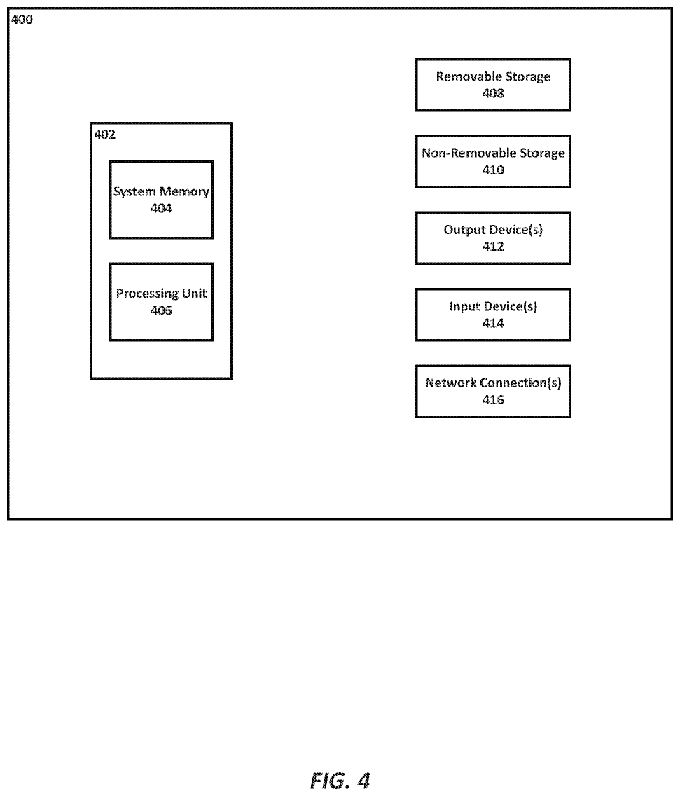

The sub-systems in the pipeline based implementation of the present disclosure are developed using python running on desktop computers. The libraries used in developing the system have mobile platform counterparts for both Android and iOS platforms are ready for deployment using respective platforms native languages. It should be understood that the computer language “python,” and the computer systems described herein are non-limiting examples, and that any computing device (e.g., the computing device 400 described with respect to FIG. 4) and software tools can be used.

Another example implementation of the present disclosure contemplates another a different strategy without the pipeline mechanism outlined above. An example design is illustrated by FIG. 3B. This implementation is a system with uses cases in the stroke market and other markets where a human operator interacts with an AI agent like the described above. The second system can use a transformer neural network (TNN) with spatial and temporal attention mechanisms to implicitly understand the organization of the body parts directly from input stream. The TNN provides scale in two stages, the first stage reasoning can provide scale scores for all the sub-systems described above which are then accumulated by a Multi-Layer Perceptron (MLP) design at the output. The TNN structure is able to adopt to multiple domains using the reference data collected for each domain. For the stroke application, the AI agent described herein provides the necessary domain data for the training of the TNN model. This disclosure contemplates that training is incremental using a reinforcement learning strategy to reduce the data collection efforts.

The example desktop implementations described herein is developed in python and uses graphics processing unit (GPU) to perform analysis. Optionally, the implementations described herein can be implemented as a mobile application, for example in Java and C++, using the same libraries the desktop version.

The porting of the existing laboratory solution to mobile platform requires a device with embedded GPU for real time patient data analysis. This disclosure contemplates potential speed differences between the current desktop solution and the mobile platform solution, which can be mitigated by implementing the stoke analysis app as a staged solution which considers each category separately to reduce the computational requirement. Additionally, the most recent tablet and phone platforms, both for iOS and Android have dedicated GPUs for Machine Learning which also mitigate speed differences. Alternatively, cloud services can be used to perform the analysis instead of running machine learning on the device. This approach sends the video clips (e.g., the sequence of images) and collected speech (e.g., the audio signal) to a cloud server which runs the algorithms to provide stroke score back to the mobile device. Again, it should be understood that implementations of the present disclosure can be implemented using any combination of servers, computers, and mobile computing devices, as described in more detail with respect to FIG. 4.

### Example Systems

An example system for detecting stroke includes an imaging device (e.g., digital video camera); a microphone; and a computing device. Imaging devices and microphones are well known in the art and therefore not described in further detail herein. Additionally, this disclosure contemplates that the computing device includes at least a processor and memory as shown in box 402 of FIG. 4. The imaging device and microphone are operably coupled to the computing device, for example, by one or more communication links. This disclosure contemplates the communication links are any suitable communication link. For example, a communication link may be implemented by any medium that facilitates data exchange including, but not limited to, wired, wireless and optical links. Example communication links include, but are not limited to, a LAN, a WAN, a MAN, Ethernet, the Internet, or any other wired or wireless link such as Bluetooth, WiFi, WiMax, 3G, 4G, or 5G.

Optionally, in some implementations, the system is a computing device (e.g., computing device 400 of FIG. 4) such as a mobile computing device, for example, a smart phone, a tablet computer, or a laptop computer. It should be understood that a mobile computing device is deployable in the field, for example, by EMTs or other medical professionals interacting with a potential stroke patient outside of the hospital setting. Alternatively, the system may be a desktop computer or other computer more permanently fixed to medical equipment and/or less mobile.

In some implementations, the operations performed by the computing device are performed locally, i.e. on resources of the system itself. Alternatively, in other implementations, the operations performed by the computing device are performed remotely, i.e. the imaging device and microphone are operably coupled to the computing device via a network such as a LAN, a WAN, a MAN, Ethernet, the Internet. Optionally, the operations are performed in a cloud computing environment.

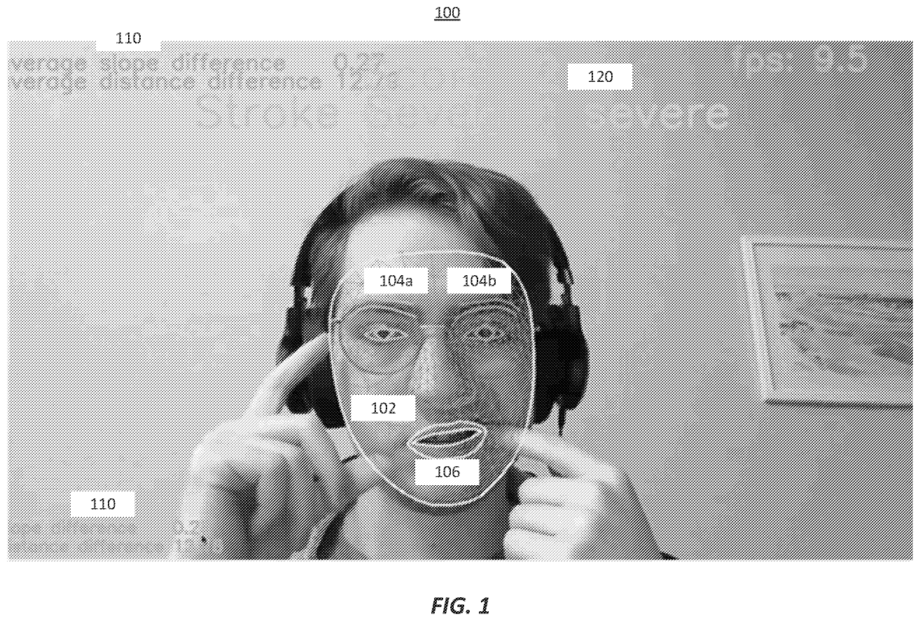

The computing device is configured to receive a sequence of images from the imaging device, the sequence of images capturing a state of a patient (e.g., posture, hands, feet, facial features); analyze the sequence of images to detect one or more of limb impairment (e.g., motor impairment sub-system described herein and/or shown in FIG. 1 and FIG. 2A), gaze impairment (e.g., facial analysis sub-system described herein and/or shown in FIG. 1 and FIG. 2C), or facial palsy (e.g., facial analysis sub-system described herein and/or shown in FIG. 1 and FIG. 2B); and assign a respective numeric score to each of the detected one or more limb impairment, gaze impairment, or facial palsy. FIG. 1 illustrates an image 100 overlaid with information. Optionally, the information shown in FIG. 1 can be generated using the systems/methods described herein, including systems and methods for scoring stroke severity and systems and methods including computer vision. A computer vision system can be used to extract features from the sequence of images. For example, the computer vision system optionally detect the face of a user and partition the face into a grid 102 (e.g., a grid of triangles). The grid can include eye locations 104a 104b and a mouth location/shape 106. The grid 102, eye locations 104a, 104b, and mouth location/shape 106 can be evaluated to determine their symmetry and/or slope. The symmetry and slope information 110 can be overlaid onto the image 100. The image 100 can be further overlaid with the stroke severity information 120 determined by the system. It should be understood that the image 100 is intended only as a non-limiting example, and that other computer vision systems and displays can be used in implementations of the present disclosure. It should also be understood that the image 100 can be one of a plurality of images (e.g., a video or video stream) and that the computer vision techniques described with reference to FIG. 1 can be combined with other methods of monitoring a medical condition, as described throughout the present disclosure.

FIGS. 2A-2D are diagrams illustrating functionality of one or more sub-systems for evaluating a patient for stroke according to implementations described herein. Optionally, the step of assigning a respective numeric scores can include using an expert system. FIGS. 2A-2D illustrate an example of rules that can be used in an expert system for assigning numeric scores. FIG. 2A illustrates motor impairment rules (e.g., whether a user can raise both hands, one hand, or neither hand). FIG. 2B illustrates facial palsy rules (e.g., eye and mouth asymmetry/droop). FIG. 2C illustrates gaze deviation rules. FIG. 2D illustrates head deviation rules. Optionally, the rules illustrated in FIGS. 2A-2D can be assigned numerical values (e.g., based on the respective levels of motor impairment, facial palsy, gaze deviation, and head deviation).

It should be understood that the expert system described with reference to FIGS. 2A-2D is only an example and that other expert systems may be used. It should be understood that the rules illustrated in FIGS. 2A-2D are non-limiting examples, and that expert systems described herein can work with different numbers, types, and combinations of rules. Alternatively, in some implementations, this disclosure contemplates that one or more deployed AI models such as machine learning models can be used to assign numeric scores. Such machine learning models may be trained using supervised or reinforcement learning strategies.

Optionally, in some implementations, the step of analyzing the sequence of images includes using a machine learning algorithm. This disclosure contemplates that raw image data and/or extracted features may be input into the machine learning algorithm. For example, the sequence of images can be analyzed using MediaPipe of Google Inc. of Mountain View, California. MediaPipe is an open source tool for building multimodal, cross platform applied machine learning pipelines. It should be understood that MediaPipe is provided only as an example tool and that other tools may be used.

The computing device is also configured to receive an audio signal from the microphone, the audio signal capturing a sound associated with the patient; analyze the audio signal to detect aphasia and/or agnosia (see aphasia/agnosia sub-system described herein); assign a respective numeric score to the detected aphasia and/or agnosia. The sound associated with the patient is the patient's voice in some implementations. For example, in some implementations described herein, the audio signal captures the patient's response to multiple questions (see agnosia analysis). Alternatively or additionally, the sequence of images captures the patient's response to multiple prompts (see aphasia analysis). It should be understood that in both of these implementations, the audio signal captures the questions/prompts. Optionally, the step of assigning a respective numeric score includes using an expert system. As described throughout the present disclosure, an expert system (e.g., rule based) can be used in some implementations of the present disclosure for assigning numeric scores. It should be understood that the expert system described herein is only an example and that other expert systems may be used. Optionally, in some implementations, the step of analyzing the audio signal includes using a machine learning algorithm. Example machine learning models include decision tree (DT) and artificial neural network (ANN). This disclosure contemplates that raw audio data and/or extracted features may be input into the machine learning algorithm. For example, as described above, the audio signal can be analyzed using MediaPipe of Google Inc. of Mountain View, California. It should be understood that MediaPipe is provided only as an example tool and that other tools may be used.

The computing device is further configured to generate a stroke score. Optionally, the stroke score can be a sum of the respective numeric scores for the detected one or more of limb impairment, gaze impairment, or facial palsy and the detected aphasia/agnosia as described above. In other words, a respective score is generated by each of the (1) motor impairment sub-system, (2) facial analysis sub-system, and (3) aphasia/agnosia sub-system, and then the respective scores are added together to obtain the stroke score. Optionally, the stroke score is a Rapid Arterial oCclusion Evaluation (RACE), National Institutes of Health Stroke Score (NIHSS), Los Angeles Motor Scale (LAMS), or Cincinnati Stroke Scale. It should be understood that RACE, NIHSS, LAMS, and Cincinnati Stroke Scale are only provided as example stroke scores and that other scores or scales, including but not limited to stroke scores approved by other organizations, may be used.

In some implementations, the computing device is further configured to diagnose the patient with a stroke based on the stroke score. This disclosure contemplates that diagnosis can optionally be based on the generated stroke score. For example, a stroke score greater than a threshold score is used for diagnosis. Optionally, the computing device is further configured to assess a severity of the stroke based on the stroke score. This disclosure contemplates that the severity of the stroke can optionally be based on the generated stroke score. For example, different ranges of stroke score are associated with different levels of severity. Optionally, in some implementations, the computing device is further configured to alert the provider based on the stroke score. Optionally, the computing device is further configured to recommend a triage action, for example, selecting an appropriate treatment facility. Alternatively or additionally, the computing device is optionally further configured to recommend a treatment for the patient. This disclosure contemplates that the computing device is optionally configured to recommend a treatment based on the stroke score alone or in combination with additional medical or clinical information for the patient (e.g., medical images such as computed tomography (CT) scan). As described above, the system is optionally mobile and can be deployed to the field (i.e., pre-hospital settings). The systems described herein provide one or more of the following improvements as compared to existing stroke scoring systems: (i) automate and standardize calculation of stroke score; (ii) reduce variations in calculation of stroke score, which may result from subjective bias and/or different levels of training/familiarity; (ii) reduce delay in providing time sensitive treatment for stroke; (iv) non-invasive; (v) inexpensive; and/or (vi) do not require expert involvement. It is well understood that treatment for stroke must be delivered quickly and within certain time limits. Treatments include, but are not limited to, administration of a thrombolytic (e.g., tissue plasminogen activator (tPA)) or performance of an endovascular procedure (e.g., EVT). In some cases, patients must be transported to specifically designated stroke centers to receive appropriate treatment (i.e. an appropriate triage action is recommended such that the patient is transferred to a center that is capable of providing care).

Another system for detecting stroke includes one or more AI models; and a computing device. The computing device is configured to receive a sequence of images, the sequence of images capturing a state of a patient (e.g., posture, hands, feet, facial features); receive an audio signal, the audio signal capturing a voice of the patient; input the sequence of images and the audio signal into the one or more AI models; and receive a stroke score, the stroke score being predicted by the one or more AI models.

Optionally, the computing device is further configured to extract one or more features from the sequence of images and the audio signal, and the step of inputting the sequence of images and the audio signal into the one or more AI models includes inputting the extracted features into the one or more AI models.

In some implementations, the one or more AI models include one or more trained machine learning models. In these implementations, the one or more trained machine learning models include a transformer neural network (TNN). Alternatively or additionally, the one or more trained machine learning models include a multilayer perceptron (MLP). Alternatively or additionally, the one or more trained machine learning models include one or more decision trees (DT). In other implementations, the one or more AI models are an expert system.

In some implementations, the one or more machine learning models are trained using supervised learning strategies. In some implementations, the one or more machine learning models are trained using a reinforcement learning strategy.

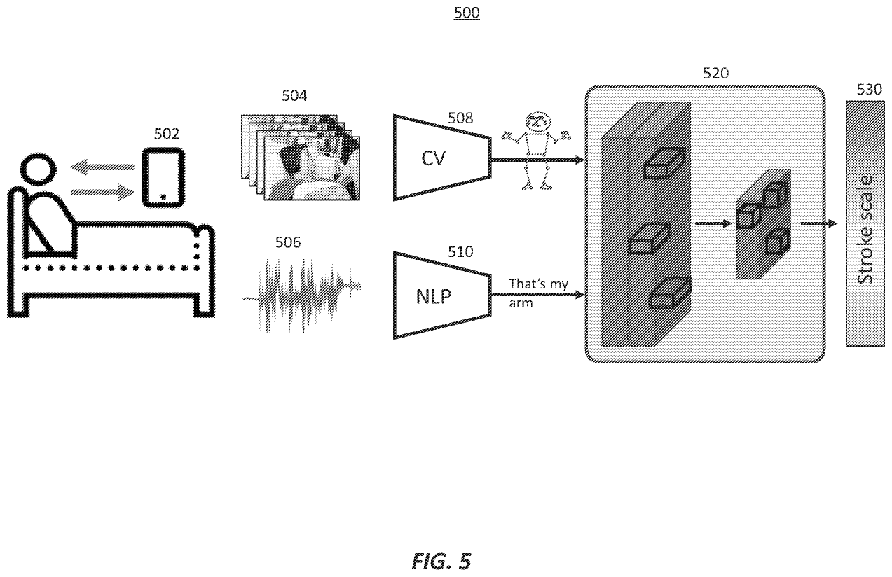

FIG. 5 illustrates a system block diagram for an example system 500 according to another implementation of the present disclosure.

Sensors 502 are configured to capture characteristics of a patient. In the example system 500, the sensors 502 collect audio signal 506 and images 504. Optionally the sensors 502 can include one or more microphones, and/or one or more imaging devices (e.g., cameras). Additionally, this disclosure contemplates that sensors 502 can optionally include devices other than a microphone and camera. For example, sensors 502 may include one or more sensors for measuring the patient's physiology (e.g., body temperature, heart rate, blood oxygen saturation, etc.). Optionally, additional sensors 502 may include sensors configured to measure a patient's psychological signs, heart rate, blood pressure, etc. It should be understood that additional sensor signals may be used to assist with the diagnosis. It should be understood that sensors 502 can be operably coupled to one or more computing devices (see e.g., computing device 400 of FIG. 4). The sensors 502 discussed above can be coupled to one or more computing devices through one or more communication links. This disclosure contemplates the communication links are any suitable communication link. For example, a communication link may be implemented by any medium that facilitates data exchange including, but not limited to, wired, wireless and optical links.

In some implementations, the system 500 can also include haptic devices (not shown) as sensors 502. As used herein, a haptic device is any device that can be used to apply a physical sensation to a patient and/or to detect a physical response of a patient. Example of physical sensations that can be applied by a haptic device are a force, a vibration, or a motion.

Numbness may be a stroke symptom, and numbness may also be a symptom of other physical and neurological conditions. Implementations of the present disclosure can detect numbness on different parts of the body (e.g., the limbs) by positioning a haptic device on a patient's body (e.g., on a limb) and using the haptic device to apply a physical stimulus to the patient. The patient can respond to the physical stimulus, or optionally not respond at all (e.g., if no physical sensation is felt, or the patient is unable to comprehend the test). Example responses include indicating that the patient feels the stimulus or indicating the patient does not feel the stimulus. The patient's responses can be captured by sensors 502 such as the microphone (e.g., verbal response) and camera (e.g., movement).

In some implementations, the system 500 can be configured to determine the patient's response to physical stimulus from a haptic device by analyzing the audio signal 506 and/or images 504 that include the patient's response to the physical stimulus. As a non-limiting example, the patient's response to the physical stimulus can be verbal, such as “yes” or “I feel it” and natural language processing can be applied to the verbal response to determine whether the response is affirmative or negative. Alternatively or additionally, computer vision can be used to determine whether a patient can feel a physical sensation from the haptic device, for example by identifying that the patient moves a limb where the haptic device is placed. Optionally, the haptic device can include sensors (e.g., an inertial measurement unit or accelerometer) configured to measure the motion of the haptic device.

Optionally, the images 504 include a sequence of images and/or a video. The images 506 can optionally be processed using computer vision techniques, which can include techniques for extracting information from images or series of images. A computer vision module 508 is illustrated in FIG. 5. Non-limiting examples of computer vision techniques that can be performed by the computer vision module 508 include scene reconstruction, object detection, event detection, video tracking, object recognition, 3D pose estimation, motion estimation, 3D scene modeling, and image restoration.

Optionally, the audio signal 506 can be processed using natural language processing (“NLP”). An NLP module 510 is illustrated in FIG. 5. Non-limiting examples of processes that can be performed by the NLP module include speech recognition, speech segmentation, and/or word segmentation.

The computer vision module 508 and NLP module 510 can be implemented using one or more computing devices (not shown). An example computing device 400 that can be used in implementations of the present disclosure is shown in FIG. 4. The computing device 400, which executes the computer vision module 508 and NLP module 510, can be operably connected to the sensors 502, for example using wired or wireless connections.

The system 500 can include an AI model 520 that can receive information from the computer vision module 508 and/or NLP module 510. Optionally, the computer vision module 508 and/or NLP module 510 are configured to extract one or more features from the images 504 and audio signal 506, respectively. Such extracted features can be input into the AI model 520. In some implementations of the present disclosure, the AI model 520 is a trained machine learning model. It should be understood that the AI model 520 can include more than one trained machine learning model. The trained machine learning model(s) can be configured to analyze a sequence of images 504 (and/or features extracted by the computer vision module 508) and/or to analyze an audio signal 506 (and/or features extracted by the NLP module 510). Based on such analysis, the trained machine learning model can generate the stroke score 530. In some implementations of the present disclosure, the AI model 520 includes one or more trained machine learning models. Optionally, the machine learning model(s) used in the present disclosure are deep learning models. For example, the machine learning model(s) used in the present disclosure can include a transformer neural network (TNN). Alternatively, the machine learning model(s) used in the present disclosure can include multilayer perceptron (MLP). In some implementations, the machine learning model(s) are supervised learning models, including but not limited to, decision trees. In other implementations, the machine learning model(s) are reinforcement learning models.

Alternatively, in some implementations of the present disclosure, the AI model 520 is an expert system. As used herein, an “expert system” refers to a computer system that emulates, replicates and/or implements a decision making ability of a human expert. An expert system can include a system that reasons through a body of knowledge using an if-then approach to derive new facts from an initial set of facts. As a non-limiting example, an expert system for stroke scoring can include a set of rules and if-then steps that can be used to analyze images/audio and determine a stroke score. It should be understood that expert systems can be used in implementations of the present disclosure in applications other than stroke scoring.

In the example implementation shown in FIG. 5, the AI model 520 can be configured to output a stroke score 530. The stroke score 530 in FIG. 5 is illustrated as a stroke scale. Optionally, the stroke score 530 can be used to diagnose a patient with a stroke, and the system 500 can output a diagnosis of a stroke with the stroke score 530. In some implementations, the system 500 can alternatively or additionally assess the severity of the stroke based on the stroke score 530 and/or output a severity of the stroke. Non-limiting examples of stroke scores 530 that can be used in the system 500 include the Rapid Arterial oCclusion Evaluation (RACE), the National Institutes of Health Stroke Score (NIHSS), the Los Angeles Motor Scale (LAMS), and/or Cincinnati Stroke Scale. It should be understood that RACE, NIHSS, LAMS and Cincinnati Stroke Scale are provided only as non-limiting example stroke scales.

The system 500 can also optionally recommend that a user or the patient take actions or receive treatment based on the stroke score 530. Non-limiting examples of treatments that can be recommended based on the stroke score 530 include administration of a thrombolytic and/or performance of an endovascular procedure. As another example, the system can recommend a triage action based on the stroke score 530 that is output. A non-limiting example of a triage action is to select an appropriate treatment facility based on a stroke score 530.

It should be understood that the system 500 illustrated in FIG. 5 can be implemented as one or more devices. For example, in some implementations of the present disclosure, the system 500 can be implemented using a smart phone, a tablet computer, a laptop computer, or a desktop computer.

### Example Artificial Intelligence and Machine Learning

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

Machine learning models include an artificial neural network (ANN), which is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a data set to minimize the cost function, which is a measure of the ANN's performance (e.g., error such as L1 or L2 loss) during training. The training algorithm tunes the node weights and/or bias to minimize the cost function. This disclosure contemplates that any algorithm that finds the minimum of the cost function can be used for training the ANN. Training algorithms for ANNs include, but are not limited to, backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

FIG. 3A is a diagram illustrating an example machine learning based pipeline for evaluating a patient for stroke according an example implementation described herein. In FIG. 3A, the image sequence is passed through a computer vision system 300 (e.g. MediaPipe) to generate feature locations 302 on the subject's body and face. Such features 302 are then passed through a special network 304 that performs differential geometric analysis of relative feature locations. The output (i.e., “stroke scale” in FIG. 3A) is generated by a regression network 306 that uses the differential features. It should be understood that a similar pipeline can be provided for the audio analysis as well. FIG. 3B is a diagram illustrating an example machine learning based pipeline for evaluating a patient for stroke according another example implementation described herein. In FIG. 3B, an end-to-end stroke scale generator network 308 includes implicit motion and audio transformers with cross modality integration scheme. It should be understood that images and audio signals may be input directly (e.g., without prior feature extraction) into the generator network 308 in this implementation. The network also includes a stroke score generator 310 in the form of a transformer regressor network for temporally consistent score generation (i.e., “stroke scale” in FIG. 3B).

### Example Methods

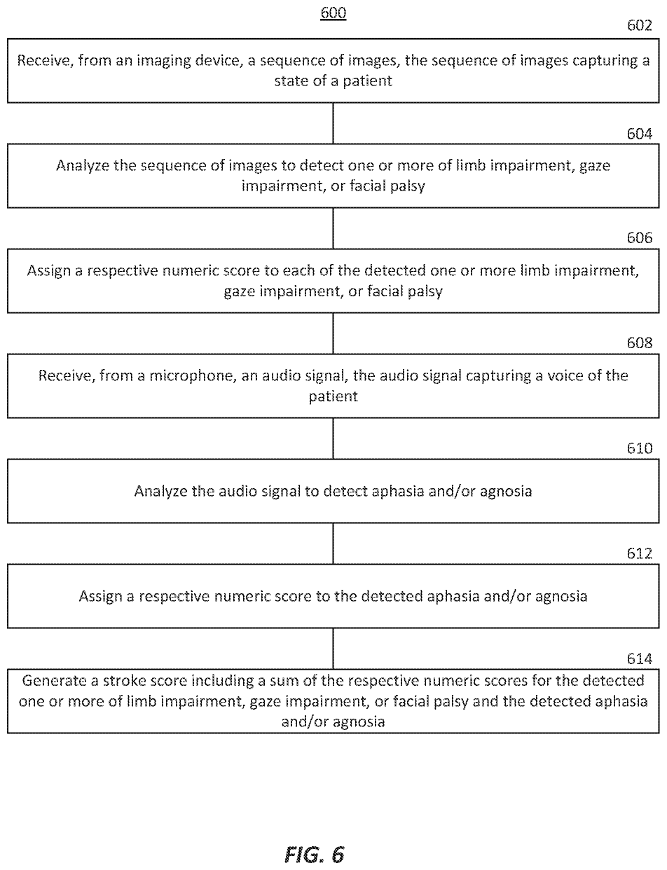

FIG. 6 illustrates example operations 600 for automated detection of a stroke that can be performed in implementations of the present disclosure. Optionally, the operations 600 can be performed by one or more computing devices (see e.g., computing device 400 of FIG. 4). Optionally, the operations 600 can be performed by a computing device that is part of the system 500 illustrated in FIG. 5.

At step 602, a sequence of images is received from an imaging device. As described herein, the sequence of images captures a state of a patient (e.g., posture, hands, feet, facial features).

At step 604, the sequence of images is analyzed. Analyzing the sequence of images at step 604 can include detecting limb impairment, gaze impairment, and/or facial palsy. Alternatively or additionally, other information can be detected in the sequence of images, including any other visual indicators of a stroke, such as gaze deviation, hemiparesis, asymmetric muscle contractions, etc.

At step 606, a respective numeric score can be assigned to each of the detected one or more limb impairment, gaze impairment, or facial palsy. Alternatively or additionally, any other information detected in the sequence of images can also be numerically scored.

At step 608, an audio signal can be received from a microphone. The audio signal can include a voice of the patient captured by the microphone.

At step 610, the audio signal can be analyzed. Analyzing the audio signal can include detecting aphasia. In these implementations, operations can include instructing the patient to perform certain tasks, and analyzing (e.g., using NLP and computer vision) the patient's response to those orders. Alternatively or additionally, analyzing the audio signal can include detecting agnosia. In these implementations, operations can include prompting the patient with questions, and analyzing (e.g., using NLP) the patient's response to those questions. Optionally, the operations include detecting both aphasia and agnosia.

At step 612, a respective numeric score can be assigned to the detected aphasia and/or agnosia.

At step 614, a stroke score can be generated. The stroke score can optionally be a sum of the respective numeric scores for the detected one or more of limb impairment, gaze impairment, or facial palsy assigned at step 606 and the detected aphasia and/or agnosia assigned at step 612.

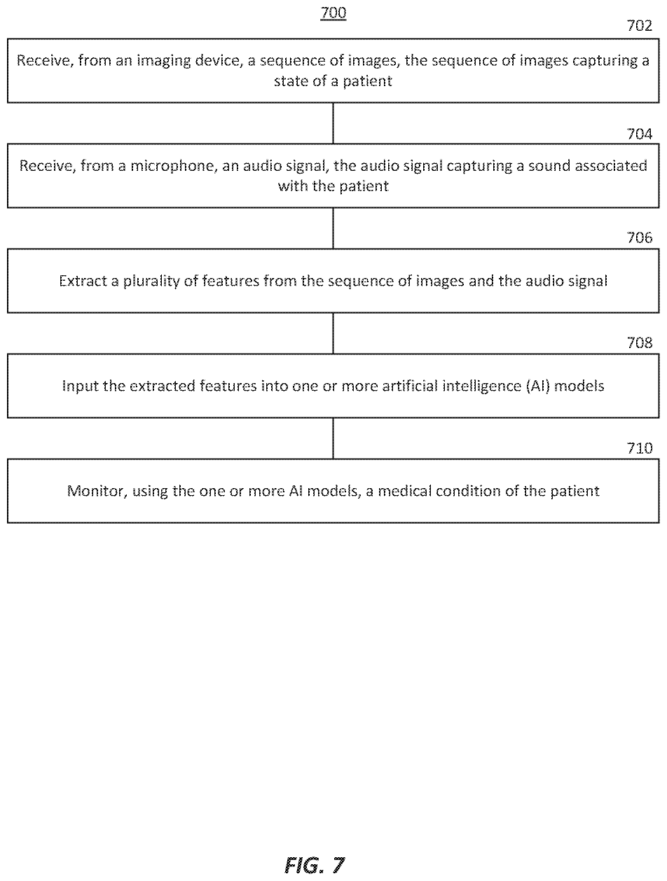

Implementations of the methods described in the present disclosure can be used to monitor and/or detect medical condition other than stroke, and the method 600 shown in FIG. 6 is intended only as a non-limiting example. With reference to FIG. 7, example operations 700 of monitoring a medical condition are illustrated. The operations 700 can be performed using one or more computing devices (see e.g., computing devices 400 of FIG. 4). Optionally, the operations 700 can be performed by a computing device of the system 500 illustrated in FIG. 5. Non-limiting examples of medical conditions that can be monitored/detected include seizure, pain management, or paralysis.

At step 702, a sequence of images is received from an imaging device, where the sequence of images can capture a state of a patient. As described herein, the sequence of images captures a state of a patient (e.g., posture, hands, feet, facial features).

At step 704, an audio signal can be received from a microphone, where the audio signal captures a sound associated with the patient.

At step 706, features can be extracted from the sequence of images and the audio signal. The features can be extracted from the images and audio signal using computer vision and natural language processing techniques, respectively, as described with reference to the computer vision module 508 and natural language processing module 510 shown in FIG. 5. As used herein, “features” extracted at step 706 can include any outputs of the computer vision module 508 and/or natural language processing module 510.

At step 708, the extracted features can be input into one or more AI models. Optionally, the AI models can include any combination of expert systems and/or trained machine learning models. The trained machine learning model used in step 708 can be a trained machine learning model according to any of the implementations described herein, including artificial neural networks, models trained using supervised or reinforcement learning strategies, and deep learning models including transformer neural networks of MIPs.

At step 710, a medical condition of a patient can be monitored using the one or more AI models. In some implementations of the present disclosure, the operations 700 can include assigning numeric scores for a medical condition. Alternatively or additionally, in some implementations of the present disclosure, the operations 700 can include diagnosis of a medical condition, recommending a triage action, or recommending a treatment based on the output of the AI models. Additionally, the operations 700 can further include recommending a triage action or treatment based on the medical condition of the patient that is determined at step 710.

### Example Computing Device

It should be appreciated that the logical operations described herein with respect to the various figures may be implemented (1) as a sequence of computer implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by dashed line 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A system, comprising:
an imaging device;
a microphone; and
a computing device comprising a processor and a memory operably coupled to the processor, wherein the memory has computer-executable instructions stored thereon that, when executed by the processor, cause the processor to:
receive a sequence of images from the imaging device, the sequence of images capturing a state of a patient;
analyze the sequence of images to detect one or more of limb impairment, gaze impairment, or facial palsy;
assign a respective numeric score to each of the detected one or more limb impairment, gaze impairment, or facial palsy;
receive an audio signal from the microphone, the audio signal capturing a voice of the patient;
analyze the audio signal to detect aphasia or agnosia;
assign a respective numeric score to the detected aphasia or agnosia; and
generate a stroke score comprising a sum of the respective numeric scores for the detected one or more of limb impairment, gaze impairment, or facial palsy and the detected aphasia or agnosia.

2. The system of claim 1, wherein the memory has further computer-executable instructions stored thereon that, when executed by the processor, cause the processor to diagnose the patient with a stroke based on the stroke score.

3. The system of claim 2, wherein the memory has further computer-executable instructions stored thereon that, when executed by the processor, cause the processor to assess a severity of the stroke based on the stroke score, and recommend a triage action.

4. (canceled)

5. (canceled)

6. The system of claim 2, wherein the memory has further computer-executable instructions stored thereon that, when executed by the processor, cause the processor to recommend a treatment for the patient.

7. (canceled)

8. (canceled)

9. (canceled)

10. (canceled)

11. (canceled)

12. The system of claim 1, further comprising an expert system, wherein the expert system is configured to analyze the sequence of images, analyze the audio signal, assign the respective scores, and/or generate the stroke score.

13. The system of claim 1, further comprising a trained machine learning model, wherein the trained machine learning model is configured to analyze the sequence of images, analyze the audio signal, assign the respective scores, and/or generate the stroke score.

14. The system of claim 1, further comprising a haptic device configured to apply a force, a vibration, or a motion to the patient, wherein the memory has further computer-executable instructions stored thereon that, when executed by the processor, cause the processor to control the haptic device.

15. The system of claim 1, wherein the stroke score is a Rapid Arterial oCclusion Evaluation (RACE), National Institutes of Health Stroke Score (NIHSS), Los Angeles Motor Scale (LAMS), or Cincinnati Stroke Scale.

16. (canceled)

17. A system, comprising:
one or more artificial intelligence (AI) models; and
a computing device comprising a processor and a memory operably coupled to the processor, wherein the memory has computer-executable instructions stored thereon that, when executed by the processor, cause the processor to:
receive a sequence of images, the sequence of images capturing a state of a patient;
receive an audio signal, the audio signal capturing a voice of the patient;
input the sequence of images and the audio signal into the one or more AI models; and
receive a stroke score, the stroke score being predicted by the one or more AI models.

18. The system of claim 17, wherein the memory has further computer-executable instructions stored thereon that, when executed by the processor, cause the processor extract one or more features from the sequence of images and the audio signal, and wherein the step of inputting the sequence of images and the audio signal into the one or more AI models comprises inputting the extracted features into the one or more AI models.

19. The system of claim 17, wherein the one or more AI models are an expert system or one or more trained machine learning models.

20. (canceled)

21. (canceled)

22. (canceled)

23. (canceled)

24. The system of claim 17, further comprising an imaging device for capturing the sequence of images and a microphone for capturing the audio signal.

25. The system of claim 17, wherein the memory has further computer-executable instructions stored thereon that, when executed by the processor, cause the processor to diagnose the patient with a stroke based on the stroke score.

26. The system of claim 25, wherein the memory has further computer-executable instructions stored thereon that, when executed by the processor, cause the processor to assess a severity of the stroke based on the stroke score, and recommend a triage action.

27. (canceled)

28. (canceled)

29. The system of claim 26, wherein the memory has further computer-executable instructions stored thereon that, when executed by the processor, cause the processor to recommend a treatment for the patient.

30. (canceled)

31. A computer-implemented method for automated detection of stroke, comprising:
receiving, from an imaging device, a sequence of images, the sequence of images capturing a state of a patient;
analyzing the sequence of images to detect one or more of limb impairment, gaze impairment, or facial palsy;
assigning a respective numeric score to each of the detected one or more limb impairment, gaze impairment, or facial palsy;
receiving, from a microphone, an audio signal, the audio signal capturing a voice of the patient;
analyzing the audio signal to detect aphasia or agnosia;
assigning a respective numeric score to the detected aphasia or agnosia; and
generating a stroke score comprising a sum of the respective numeric scores for the detected one or more of limb impairment, gaze impairment, or facial palsy and the detected aphasia or agnosia.

32. The computer-implemented method of claim 31, further comprising diagnosing the patient with a stroke based on a stroke score.

33. The computer-implemented method of claim 32, further comprising assessing a severity of the stroke based on the stroke score.

34. The computer-implemented method of claim 32, further comprising recommending a triage action.

35. The computer-implemented method of claim 32, further comprising recommending a treatment for the patient.

36-45. (canceled)
